(19) United States
(12) Patent Application Publication
(10) Pub. No.: US 2022/0370713 A1
(43) Pub. Date: Nov. 24, 2022

(54) INFUSION CONTROLLER USING INLINE FEEDBACK THROUGH INTEGRAL FLOW MEASUREMENT IN TUBING

(71) Applicant: INNOVATIVE HEALTH SCIENCES, LLC, Chester, NY (US)

(72) Inventor: Andrew SEALFON, Chester, NY (US)

(21) Appl. No.: 17/761,414

(22) PCT Filed: Sep. 18, 2020
(86) PCT No.: PCT/US2020/051592

§ 371 Date: Mar. 17, 2022

Related U.S. Application Data
(60) Provisional application No. 62/902,584, filed on Sep. 19, 2019.

(51) Int. Cl. A61M 5/168; A61M 5/142; A61M 5/162

(52) U.S. Cl. CPC A61M 5/16804; A61M 5/142; A61M 5/162; A61M 5/16886; A61M 2205/502

(57) ABSTRACT

An administration set, system, and method for administering an infusion fluid for delivering an infusion fluid into a patients anatomic space includes an infusion fluid path having a first portion to connect with a source of the infusion fluid and a second portion to deliver the infusion fluid into the patients anatomic space; and a flow rate sensor disposed in the infusion fluid path between the first and second portion to determine a flow rate measurement of the infusion fluid and to generate a signal indicative of the flow rate measurement to control the flow rate of the infusion fluid through the fluid path.

### TECHNICAL FIELD

This invention relates generally to infusion pump systems, and more particularly, to infusion pump systems and methods of delivering infusion fluids with a flow rate measurement and control system built into the administration set tubing.

### BACKGROUND

An infusion pump is a medical device that delivers fluids, including nutrients and medications, into a patient in controlled amounts. The nutrients and medications can include insulin, other hormones, antibiotics, chemotherapy drugs, pain relievers, and other fluids. Infusion pumps can be used to deliver fluids intravenously, as well as subcutaneously (beneath the skin), arterially, and epidurally (within the surface of the central nervous system). Infusion pumps can reliably administer fluids in ways that would be impractically expensive, unsafe, or unreliable if performed manually by a nursing staff. Infusion pumps offer advantages over manual administration of fluids, including the ability to deliver fluids in very small volumes and the ability to deliver fluids at precisely programmed rates or automated intervals. For example, infusion pumps can administer 1 ml per hour injections (too small a dose for a drip), injections every minute, injections with repeated boluses requested by the patient, e.g., for patient-controlled analgesia (up to a maximum number allowed over a time period), or fluids whose volumes vary by the time of day.

Infusion pump systems often use disposable infusion sets to link the pump system to an infusion site of a patient. These sets usually have tubing between the infusion site and the infusion pump. For constant flow pump systems, the tubing is referred to as an extension set with undefined flow properties. In other words, the extension set is not intended to control, limit, or set the flow rate in any way. For mechanical {pressure} pumps, the tubing generally needs to be selected to create the desired flow rate requirements of drugs, which specifically includes the influence of fluid viscosity.

Infusion pumps are frequently used to administer critical fluids, including high-risk medications, so pump failures can have significant consequences for patient safety. Many infusion pumps are fitted with safety features, including alarms and other operator alerts, that activate in the event of a problematic incident. Some pumps alert users when air or another obstruction is detected in the tubing that delivers the infusion fluid to the patient. Smart infusion pumps alert the user when there is a risk of an adverse drug interaction, or when the user programs the operating parameters outside of specified safety limits.

While some adverse treatment events may be the result of user error, many of the reported adverse events are related to deficiencies in infusion system design and engineering. These deficiencies create problems themselves or contribute to user error. The most common types of reported problems have been associated with software defects, user interface issues, and mechanical or electrical failures. Software defects often result in displayed error messages in the absence of an identifiable problem or incorrect interpretation of infusion rates due to key bounce. User interface errors include confusing screen messages, units of measurement entries, improper cleaning, maintenance, and false alarms.

Other uses of infusion pump systems can cause discomfort or safety concerns for the patient due to excessive pressure in the system, such as when an obstruction in the infusion fluid path is encountered or when the infused fluid fills and saturates the anatomic space. A deficiency of current electronic infusion pump systems is their immediate increase in pump pressure to compensate for resistance within the infusion circuit (vein blockages, any obstructions anywhere in the fluid path, tissue saturation, etc.). This deficiency, especially due to combinations of these obstructions, results in high fluid pressure and patient harm. For example, if a problem occurs in a fluid circuit, such as a blockage in a connector, an electric pump increases pressure to overcome the blockage to deliver the desired flow rate. If a blockage in a vein subsequently occurs, the increased pressure that the pump provided to overcome the first blockage will be delivered to the vein with disastrous consequences to the patient.

One of the problems with conventional infusion pump systems is the inability to accurately measure pressures and identify conditions that affect the infusion rate and safely respond to act within the patient's anatomic space.

Most infusion pumps operate on the principle of a constant flow rate set by mechanical principles. These systems base indications of the actual flow rate of fluids delivered to the patient on indirect determinations. For example, electric pumps that are programmed to create a flow rate can deliver a specific flow rate only when there are no restrictions or blockages. If a slow flow rate is programmed into an electric pump, and the output to the patient is completely blocked off, the electric pump system will continue to indicate that specific pre-set flow rate, regardless of the actual flow rate that is being delivered to the patient. This indicates that the pump continues to build up pressure in efforts to achieve the specified flow rate. During this time the patient may, in fact, be receiving no volume of drug as a result of a total blockage. The inability to detect this change in delivered flow rate that the patient is actually receiving contributes to patient site reaction and potentially dangerous outcomes. If, for some reason, the blockage is suddenly cleared, the higher pressure will deliver a sudden bolus to the patient while the flow rate on the (infusion pump) display will continue to display the pre-set flow rate that was programmed. This can result in patient harm. This process can continue to occur in cycles as these electric pumps do not indicate the actual flow rate under many circumstances. Additionally, there are currently no available infusion pumps that can measure the flow rate directly being delivered to the patient by means of connective tubing. Similarly, other infusion systems, as described below, also indirectly measure the actual flow rate of fluid that the patient is receiving resulting in the same issues.

Gravity Systems: Gravity infusion systems have been used for more than 100 years. In gravity systems, a fluid container is positioned above the patient and a means of pinching the tubing line or otherwise adjusting the flow rate of the fluid is provided along with an indication of the flow rate. For example, a drip chamber is often used in efforts to set a particular flow rate. Gravity systems are notorious for having poor flow rate accuracies, with errors ranging from +100% to 0% after system set up. The specific location of the flow control roller clamp (i.e., a flow rate control) plays a role in this accuracy along with the height of the fluid reservoir and the height of the fluid relative to the infusion site. These factors all affect the flow rate because of the pressure difference. Some companies have designed flow controllers that adjust flow for pressure differences. One example of such a controller is the Baxter Control-A-Flo. These designs improve the performance over roller clamps but are more expensive and still result in variability of the flow rate, limiting the applications where they can be used. Gravity infusion-driven deliveries are still common in many hospitals and sub-acute facilities because of their relatively inexpensive costs.

Electrically powered infusion systems: Current medical infusion pump systems that are electrically driven operate on the same principles used 100 years ago. In current systems, drugs are typically titrated into known masses (milligrams) of drug per volume (milliliter), and the electric infusion pump is then programmed to deliver a desired flow rate of drug. The only consideration is to preset the flow rate into the pump. The exact mechanisms vary (e.g., a syringe pump with hypoid gears, or a peristaltic pump with rollers or fingers) but have the common theme that once the flow rate is set, the pump volumetrically attempts to deliver a specified flow rate (ml/hr) of the fluid. Pumps that operate in this fashion are referred to as constant rate pumps. One disadvantage with these pumps is that the pressure is usually allowed to float to maintain the specified flow rate, and in some cases can be quite high, above 2 Bar and as high as 5 bar. These high pressures create a series of problems for patients, such as tissue necrosis from infiltration or extravasation at high pressures. For children, this can result in peripheral limb amputation in serious cases. Many electric pumps have alarms to indicate when the pressure reaches a preset maximum. These “occlusion” alarms are taking pressure measurements to determine when the measured pressure reaches the preset maximum. Once met, the infusion is halted as the alarm activates. Several additions have been made with these electric volumetric pumps such as differential pressure measurements, but the basic concept of constant rate delivery has inherent over-pressure complications that are not mitigated by existing designs. It should be noted that 30% to as high as 60% of peripheral infusions result in site complications. Although the electric pump does not cause these site complications, the increasing pressure can contribute to significant adverse events for a patient and is therefore undesirable.

Constant Pressure Infusion Systems (CPS): Constant pressure infusion systems are mechanical in design and operate using a constant pressure driver acting on all the series tubing within an administration set to create a desired flow rate that is delivered to the patient's anatomic space. Constant flow systems have inherent advantages such as limited and safe pressures, and flow rates that start at the desired initial flow rate. But if any obstruction or resistance occurs inside the patient, the constant pressure systems will automatically and immediately decrease the flow rate, adjusting to the patient tissue saturation or other resistance. Great care must be taken to ensure that the entire fluid path is parametrically well defined to set an initial flow rate. More viscous fluids need consideration with these systems as well as routes of administration that involve long lengths of small diameter tubing. Everything in the fluid path must be considered in the initial flow rate determination and setting process. There is a definite safety advantage with CPS because the flow rate is reduced in response to patient site delivery restrictions to help ensure that over-delivery of the drug is minimized. The flow rate will eventually reach an equilibrium based on the pressure at the sites and the constant pressure of the pump.

The limitations of CPS are that the actual flow rate for an entire infusion is difficult to determine because of any blockage or restriction that may occur during the administration. Further, drug companies require known flow rate deliveries to receive FDA approvals for drugs. Since there is no direct measurement or indication of the actual flow rate in the current CPS, drug companies cannot correctly claim a certain drug flow rate during clinical trials with these devices. Setting different flow rates is also more complicated, as a different precision extension set is generally required to be paired with needle sets for each different drug flow rate desired. Further, some means of a flow rate calculator or a flow rate chart may be required to determine the flow rate for different drugs and combinations of ancillary delivery hardware. Flow rate calculators and charts can be confusing to use and are prone to user error, resulting in incorrect flow rates.

Other Mechanical Infusion Pumps, Coil Spring, Elastomeric: The coil spring pump is commonly referred to as a variable pressure systems (VPS) because the pressure at the beginning of an infusion syringe when the syringe pump contains a fully loaded syringe will be higher than at the end of the infusion when the syringe empties. The Hagen-Poiseuille equation, a fluid dynamics law that can be used to determine flow rate, directly relates the flow rate as a function of pressure, given certain assumptions. From this, higher pressures yield higher flow rates. In VPS systems pressure variation is not measured. Thus, the flow rate becomes indeterminate and cannot be estimated, because if anything interferes with the flow rate delivery to the patient, the pump responds by decreasing the flow rate as a function of volume and subsequently time. This is a disadvantage of the VPS design compared to the CPS, because at every volume there will be a new and different equilibrium, resulting in performance variability.

An elastomer pump uses a balloon-like structure which is filled with a drug and then connected to the patient through an administration set similar to the CPS or VPS. The elastomer pump has the advantage of being disposable after use, and thus may be simpler for the patient. Disadvantages of elastomer pumps are that they can be highly inaccurate, and the flow rate during the infusion tends to vary greatly. These performance variations occur from pump to pump, as well as from balloon filling and use time, and temperature effects, such as a chilling/freezing (used for some drugs). Elastomer pumps also tend to trap large amounts of drug, and are also known to trap air and require the user to remove the air prior to use.

The above information disclosed in this Background section is only for understanding of the background of the inventive concepts, and, therefore, it may contain information that does not constitute prior art.

### SUMMARY

Infusion systems constructed according to the principles and exemplary implementations of the invention address one or more of the above-noted drawbacks and disadvantages by incorporating a flow rate sensor into one or more extension or administration sets to determine instantaneous flow rate information, which may then relayed back to the controller unit to set and control the flow rate. With the ability to instantaneously measure the real-time flow rate being delivered to the patient, significant improvements in safety, accuracy, and performance can be achieved at a low cost. The flow rate may be controlled by a gating system and method that automatically controls the flow rate by opening and closing the gates, by changing the pulse width, and by changing the number (frequency) of the pulses. The gating system may also simultaneously provide delivery parameters about the infused fluid.

Infusion systems constructed according to the principles and some exemplary implementations of the invention sense blockages, air in the fluid line, and total fluid delivered. They can be used with many pressurized pumping mechanisms (e.g., elastomeric pumps, peristaltic pumps, and other types of pumps that deliver fluids).

Accordingly, infusion systems constructed according to the principles and some exemplary implementations of the invention incorporate a direct measurement of the actual fluid flow rate, and then adjust the system (pump) to deliver the flow rate required. This is a major safety advantage as the pump and fluid pressures are significantly lower than existing systems, and the adjustment of the flow rate can be made independent of the pump pressure up to the maximum flow rate for the fluid and route of administration used. The choice of pressure systems available can be selected from gravity pressures, elastomer pressures and up to higher pressure pumps that may be required to deliver viscous solutions at high flow rates through different routes of administration.

Some exemplary implementations of the invention include a dedicated administration or extension set incorporating flow rate sensor(s) and a digital flow controller unit to control the fluid flow by a series of (pressurized) pulses of discrete amounts of the fluid. The fluid flow rate can be controlled by opening and closing the gates for a period of time, thereby allowing the maximum flow rate when the gates are open, and turning off the flow rate when the gates are closed. By controlling the timing of the opening and closing of the gates, any flow rate (i.e., volume per time) up to the maximum (“gates open all the time”) flow rate can be achieved. For example, with 10% duty cycle (i.e., gates open 10% of the time), the flow rate is about 10% of the maximum flow rate. At 90% duty cycle, the flow rate is about 90% of the maximum flow rate. Similarly, changing the pulse width (as opposed to changing the pulse frequency) can also be used to control the flow rate. Thus, the actual fluid flow rate delivered to the patient is always monitored and controlled. The desired flow rate may be programmed into a fluid flow controller, and the detected flow rate measurement fed back to the flow controller. The flow controller can alter the pulse rate and width to generate and maintain the desired flow rate. In the event that back pressure is encountered inside the patient (e.g., vein blockage for intravenous or tissue saturation for subcutaneous administration) then either the pulse width can be increased to allow more drug to flow, or the pulse rate can be decreased in accordance with the desires of the health professional. In both cases, the maximum pressure is limited to a preset maximum pressure that can be determined as a safe maximum limit.

In one exemplary implementation of the invention, the flow rate sensor may be a thin nano wire of platinum surrounded by thin layer of parylene, an FDA approved coating to isolate the infusion fluid from electrical effects. This configuration is sensitive to deflection which is nearly directly proportional to flow rate (after compensating for temperature and viscosity). Kinematic viscosity may be determined by using the pulse or gating technique and by measuring the settling time. An estimate of dynamic viscosity may be made with the use of two sensors in series in the tubing, taking an initial reading of flow rate and then making further calculations of density and viscosity as required. The sensor can also detect air in the system and provide appropriate alarms. These devices can be fabricated using silicon wafer solid state techniques which are reliable and deliver sufficient volumes to be cost effective. In other exemplary embodiments, other sensors may be used to measure settling time during gate shut off of a pulse. However, because the settling time may be very short, the sensor must be capable of high-speed measurement of flow rate or pressure.

Another exemplary implementation of the invention creates a temperature increase to the fluid and measure the downstream effects. Additionally, another exemplary implementation of the invention includes an assessment of flow rate that is determined by a spinning wheel arrangement built into the dedicated extension set. The spinning wheel arrangement has magnetic sensors and uses Hall effect devices to sense wheel rotation which is then converted to flow rate in ml/hr.

Some exemplary implementations of the invention incorporate a gating device and technique to generate the pulses. The gating device interrupts the flow of fluid into discrete pulse widths and lengths to create a range of flow rates from very slow to the fastest open flow rate that the system can deliver. In one exemplary implementation of the invention, there are two gates spaced at a pre-determined distance. The two gates provide a safety improvement where, if any gate becomes inoperative, the second gate can be used to prevent excessive drug delivery. The two-gate design also improves low flow rate adjustments, as the tubing itself can be used to create very slow flow rates by opening the upstream gate with the downstream gate closed. Then, by opening the downstream gate and closing the upstream gate, a small amount of drug is released that was stored in the tubing between the two gates. The gates may be used to shut off the in-line flow on the tubing, so each tubing includes a soft tubing area for these gates to act upon. The gates themselves may use a positive toggle action switch which requires very low energy (electrical consumption) but decisively shuts off the fluid flow. The control of the gates may be under microprocessor control which includes the feedback from one or more flow sensors that detect the flow rate and send the appropriate gating commands to the gates (or switches) to ensure the output matches the flow rate desired.

Double sensors constructed according to the principles and some exemplary implementations of the invention may be provided for initial dynamic viscosity and confirming flow rates. While a single flow rate sensor might be sufficient for some applications, there are specific advantages for using two flow rate sensors (note—this is not to the same as the two-gate system above). Using two sensors provides an estimation of the dynamic viscosity of the infusing fluid on start-up, as the fluid impinges on the first sensor and is then timed to reach the second sensor. The time difference is the first estimate of wide-open flow rate in the system. The gating method can be used to estimate the kinematic viscosity as outlined above. This is accomplished by gating off and then on, and determining the acceleration settling time based on Stokes' law. The settling time and kinematic viscosity of a fluid abruptly turned off can be determined based on frictional forces, the diameter (or radius) of the tube, and the flow velocity. With sufficiently quick response time in the sensors, and somewhat rigid tubing to obtain clean signals, it is possible to determine the kinematic viscosity from a fluid which is used to determine the actual flow rate of the fluid without knowing the viscosity in advance.

Double sensors constructed according to the principles and some exemplary implementations of the invention also detect air in the line and end-of-infusion indications. The sensors react to restrictions occurring inside the patient which cause the fluid flow rate to decrease. However, if the clinician wanted to set the fluid flow rate at a certain value, then the gating method can change the pulse configurations to compensation and to maintain the fluid flow rate (up to the maximum fluid flow rate created by the system pressure). System pressure can be increased for viscous fluids meeting internal patient resistance.

Additional features of the inventive concepts will be set forth in the description which follows, and in part will be apparent from the description, or may be learned by practice of the inventive concepts.

According to one aspect of the invention, an administration set for delivering an infusion fluid into a patient's anatomic space includes an infusion fluid path having a first portion to connect with a source of the infusion fluid and a second portion to deliver the infusion fluid into the patient's anatomic space; and a flow rate sensor disposed in the infusion fluid path between the first and second portion to determine a flow rate measurement of the infusion fluid and to generate a signal indicative of the flow rate measurement to control the flow rate of the infusion fluid through the fluid path.

The sensor may include two flow rate sensors spaced apart a predefined distance within the infusion fluid path.

The administration further may include an extension set; and a needle tube, and one of the flow rate sensors may be disposed in the extension set and another flow rate sensor may be disposed in the needle tube, with the sensors being spaced apart a predefined distance.

The administration further may include a second flow rate sensor, and the flow rate sensors may be disposed in the infusion path and may be spaced apart a predefined distance within the infusion fluid path.

The sensor may be selected from one of the group of a nano wire of platinum coated with a thin layer of parylene, a spinning wheel sensor, and a platinum wire strain sensor, and the flow rate measurement may include a resistance measurement.

The administration further may include a controller to receive the signal indicative of the flow rate measurement from the flow sensor, and parameters input by a user, where the parameters may include at least one of the group of a pre-set maximum pressure, a pre-set resumption pressure, and a pre-set flow rate; and a fluid flow gate to control the flow rate of the infusion fluid through the fluid path.

The controller may be a programmable logic controller configured to operate the fluid flow gate to control the flow rate of the infusion fluid through the fluid path in a pulsatile manner.

The fluid flow gate may include at least two gates configured to turn on and off, and the flow rate sensor disposed in the infusion path may be configured to generate a signal indicative of the flow rate measurement to enable the controller to determine at least one of the group of a viscosity, a presence of air inside the fluid path, and a flow rate and error correction.

The signal received from the sensor may be used to determine internal pressures inside the patient's anatomic space to limit adverse site reactions.

The controller further may be configured to generate an alarm signal when the flow rate exceeds a maximum flow rate, or when the flow rate fails to meet a minimum flow rate, or when the controller acts upon the fluid flow gate to stop or slow flow of the infusion fluid for patient safety.

According to another aspect of the invention, an infusion system for delivering an infusion fluid into a patient's anatomic space includes: a pump to drive infusion fluid from a source of the infusion fluid into the patient's anatomic space; and an administration set including: an infusion fluid path having a first portion fluidically connected to the pump and a second portion to deliver the infusion fluid into the patient's anatomic space; and a flow rate sensor disposed in the infusion fluid path to determine a flow rate measurement of the infusion fluid and to generate a signal indicative of the flow rate measurement to control the flow rate of the infusion fluid through the fluid path.

The infusion system further may include: a fluid flow gate to control the flow rate of the infusion fluid through the infusion fluid path; and a controller to control at least one of the group of the pump and the fluid flow gate based upon the signal from the flow rate sensor, and where the controller may be configured to modulate the flow rate of the infusion fluid including to reduce an instantaneous flow rate to zero or to maintain an actual pressure of the infusion fluid within a predetermined acceptable range.

The controller may be configured to modulate a flow rate of the infusion fluid based on the signal using a pulse width modulation to create an off/on cycle to control the flow rate of the infusion fluid, and the pulse width may include a time in which the infusion fluid is flowing at a non-zero flow rate to maintain an actual pressure of the infusion fluid within a predetermined acceptable range.

The in-line flow rate sensor may include at least one of the group of a nano wire of platinum coated with a thin layer of parylene, a spinning wheel sensor, and a platinum wire strain gauge, and the flow rate measurement comprises a resistance measurement.

The infusion system may further include: an extension set; and a needle tube, and the flow rate sensor may include two flow rate sensors, with one of the flow rate sensors disposed in the extension set and another flow rate sensor disposed in the needle tube, with the sensors being spaced apart a predefined distance.

The infusion system further may include: a second flow rate sensor, and the flow rate sensors may be disposed in the infusion path and may be spaced apart a predefined distance.

The pump may include an infusion driver selected from one of the group of a constant pressure system, elastomeric pump, gravity system, coil spring pump, variable pressure pump, and electrically powered pump.

According to another aspect of the invention, a method for administering an infusion fluid into a patient's anatomic space via an infusion fluid path includes: receiving an in-line flow rate measurement from an in-line flow rate sensor in the infusion fluid path; modulating a flow rate of the infusion fluid into the patient's anatomic space to maintain the in-line flow rate measurement within a predefined acceptable range.

The method further may include: determining at least one of the group of a viscosity, a presence of air inside the fluid path, and a flow rate; and error correcting to maintain the flow rate.

The method further may include: determining, based on a signal from the sensor, internal pressures of the patient's anatomic space; and adjusting the flow rate of the infusion fluid to limit adverse site reactions.

The step of receiving the in-line flow rate measurement may include receiving a resistance measurement from at least one of the group of a nano wire of platinum coated with a thin layer of parylene, a spinning wheel sensor, and a platinum wire strain gauge.

The step of modulating the flow rate of the infusion fluid into the patient's anatomic space may include: changing at least one of the group of a pulse repetition frequency of the infusion fluid, a duration (width) of a pulse of the infusion fluid, a pulse width of the infusion fluid, and a period of a pulse of the infusion fluid.

The infusion fluid may be a chemotherapeutic, antibiotic, or an immunoglobulin.

It is to be understood that both the foregoing general description and the following detailed description are exemplary and explanatory and are intended to provide further explanation of the invention as claimed.

The accompanying drawings, which are included to provide a further understanding of the invention and are incorporated in and constitute a part of this specification, illustrate exemplary embodiments of the invention, and together with the description serve to explain the inventive concepts.

### BRIEF DESCRIPTION OF THE DRAWINGS

FIG. 1 illustrates an exemplary embodiment of an infusion system constructed according to the principles of the invention for delivering immunoglobulins to patients as part of their infusion therapy.

FIG. 2 illustrates another exemplary embodiment of an infusion system constructed according to the principles of the invention for delivering immunoglobulins to patients as part of infusion therapy.

FIG. 3 is a table summarizing the features and advantages of an exemplary embodiment of an infusion system constructed according to the principles of the invention in comparison to certain conventional systems.

### DETAILED DESCRIPTION

In the following description, for the purposes of explanation, numerous specific details are set forth in order to provide a thorough understanding of various exemplary embodiments or implementations of the invention. As used herein “embodiments” and “implementations” are interchangeable words that are non-limiting examples of devices or methods employing one or more of the inventive concepts disclosed herein. It is apparent, however, that various exemplary embodiments may be practiced without these specific details or with one or more equivalent arrangements. In other instances, well-known structures and devices are shown in block diagram form in order to avoid unnecessarily obscuring various exemplary embodiments. Further, various exemplary embodiments may be different, but do not have to be exclusive. For example, specific shapes, configurations, and characteristics of an exemplary embodiment may be used or implemented in another exemplary embodiment without departing from the inventive concepts.

Unless otherwise specified, the illustrated exemplary embodiments are to be understood as providing exemplary features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an exemplary embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the exemplary term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various exemplary embodiments are described herein with reference to sectional and/or exploded illustrations that are schematic illustrations of idealized exemplary embodiments and/or intermediate structures. As such, variations from the shapes of the illustrations as a result, for example, of manufacturing techniques and/or tolerances, are to be expected. Thus, exemplary embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

Infusion systems and methods constructed according to the principles and exemplary embodiments of the invention accurately and reproducibly deliver an infusion fluid to a patient at a desired anatomical location by including a flow rate measurement system built into the administration set tubing to accurately and instantaneously measure flow rate, viscosity and other parameters of the infusion fluid in the tube(s) of the administration set and to provide immediate feedback to the infusion pump to control flow rate parameters.

According to one or more exemplary embodiments of the invention, the infusion system includes a fluid reservoir (e.g., an infusion fluid storage device such as a syringe), an infusion fluid (such as a chemotherapeutic fluid or an immunoglobulin), an infusion driver (often a pump), and an administration set that is in fluid contact with the fluid reservoir and can be inserted into the patient's anatomic space or connected to deliver fluid to the patient's anatomic space. In one exemplary embodiment of the invention, the systems include a flow rate sensor in the tubing connected to the administration set. The flow rate sensor determines a flow rate and, in the case of a decreased flow rate, identifies an increased resistance and/or pressure at the site, causing the decreased flow rate. The sensor measurements of the infusion fluid in real-time are the flow rate feedback sent to a controller computing unit. The controller receives the flow rate feedback and computes a site pressure. If the calculated site pressure is too high, the controller continues to monitor and regulate the flow rate and flow characteristics (pulse modulation) of the infusion fluid. to regulate and/or maintain the site pressure. The flow rate sensor is positioned in the administration set tubing as an in-line sensor between the pump and the end of the administration set. In one exemplary implementation of the invention, the sensor is located between the fluid infusion pump and an administering end of the administration set (i.e., the end of the administration set closest to the patient's anatomical site). The in-line sensor measures the flow rate of the infusion fluid and provides the measurements to the controller. If the infusion fluid is not flowing correctly due to an occlusion or increased resistance, the system pressure increases (within preset safe pressure bounds) to compensate, and the controller can then alter the characteristics of the fluid flow (pulse modulate, decrease flow rate, etc.) or provide a warning to the patient or clinician or other user that the pressure at the patient's infusion anatomic site has exceeded a maximum pre-set value due to an occlusion or other event in which they device may be shut off.

Infusion systems constructed according to the principles and exemplary embodiments of the invention provide a safe and effective system for delivering a rapid infusion fluid flow rate using a fluid reservoir, an infusion fluid, an infusion pump, and an administration set to deliver the infusion fluid from the reservoir into the patient (via the administration set). The system may include a controller that monitors the flow rate, calculates the pressure of the infusion fluid at the anatomic site, and controls the flow rate of the infusion fluid, by adjusting the pulse to maintain a specified flow rate within an appropriate tolerance. As outlined above, when a gate is in a fully-opened state, the flow is at a maximum flow rate (when the site pressure is 0 psi), and when the gate is in a closed state, the flow is off. If the gate is pulsed with a 50% duty cycle (i.e., half the time open and half the time closed), the flow rate is half of the maximum flow. Intuitively, at 10% duty cycle, the flow rate is 10% of the maximum flow, and at 90% duty cycle, the flow rate is 90% of the maximum flow.

The flow rate can be set to a pre-set value and held constant during the infusion (if the site pressure remains within the pre-set range (0-1.0 psi) or can be varied (decrease) during the infusion if the calculated site pressure is below the pre-set maximum site pressure. The flow rate can be set to a pre-set value and will decrease in response to increasing site pressures based upon the differential pressure (AP). Alternatively, the decreased flow rate may be compensated to return to the pre-set flow rate value by increasing the pulse modulation (width, pulse repetition frequency, duty cycle, etc.) up to the maximum pressure range or the pressure tolerable at the patient site. For example, gate timing may be held constant such that as the flow rate decreases the pressure at the patient site may be calculated. Additionally, the gate pulse duty cycle may be increased to compensate for increased resistance at the infusion site to make sure pressure at the infusion site remains within safe limits, and the system continues the pre-set flow rate of infusion.

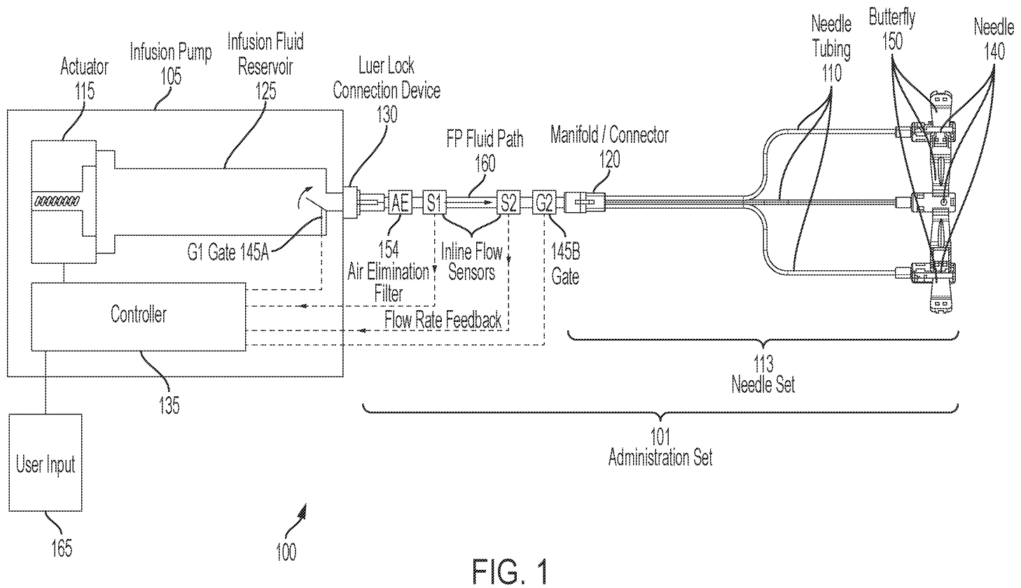

FIG. 1 illustrates an infusion system 100 constructed according to an exemplary embodiment of the invention for delivering immunoglobulins to patients as part of their infusion therapy. The infusion system includes an infusion pump (e.g., driver) 105, which is fluidically connected to an administration set 101.

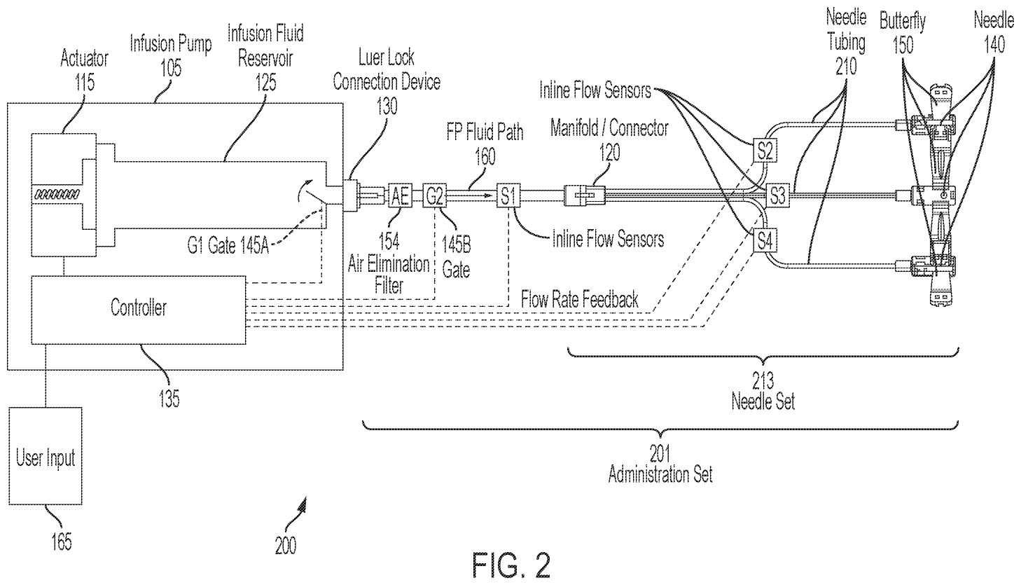

The infusion pump 105 may include an infusion fluid reservoir 125 and actuator 115. The actuator may be one of the below described drivers, including: gravity-controlled drivers, elastomeric pumps, constant pressure systems (CPS), variable pressure systems, and coiled spring pumps. In the exemplary embodiment of the infusion system 100 illustrated, the actuator 115 of the infusion pump 105 is a coiled spring pump actuator, but other known infusion pumps may be used, such as infusion pumps of the type disclosed in the assignee's U.S. patent application Ser. No. _____ entitled “Systems and Methods for Precision Matched Immunoglobulin Infusion” (Attorney docket no. 213.0001-US00), filed simultaneously herewith, the disclosure of which is incorporated herein in its entirety. In some exemplary embodiments, as illustrated in FIGS. 1 and 2, the infusion pump may also include a controller 135 and flow switch (gate) 145A. Further, the infusion pump 105 may be configured to generate flow rates between 0.5 ml/hr to 300 ml/hr, which may result in pressures at an infusion site between 0.3 psi up to 3.0 psi for subcutaneous uses, and between 0.3 psi up to 15 psi for intravenous uses. This fluid flow controller 135 may be used for all infusion pump applications from gravity to pressure systems.

The administration set 101 may include flexible tubing, extension sets, needle sets and/or other structure defining a fluid path to deliver infusion fluids from the pump 105 to a patient. The administration set 101 may include an extension set 160 fluidically connected to a connector/manifold 120. In subcutaneous uses, the administration set 101 can include a needle set 113 including a manifold/connector 120, needle tubing 110, and the needle 140. The needle set 113 may include a subcutaneous butterfly administration set 150 and one or more needles 140 to deliver an infusion therapy subcutaneously, such as immunoglobulins to patients. In intravenous uses, the extension set 160 does not use a needle set 113, and may simply include extension set 160. The extension set 160 may deliver an infusion therapy for intravenous delivery to a patient, such as delivering an antibiotic via an indwelling catheter.

The extension set 160 and needle set 113 may further include one or more in-line flow rate sensors S, such as S1 and S2 shown in FIG. 1, one or more flow switches (gates) 145, and air elimination filter(s) 154, where the gates 145A and 145B sandwich the sensors S1 and S2. Other extension sets 160 may include varied sensor S and gate 145 series ordering. In some exemplary embodiments, the sensors S1 and S2 may sandwich the gates 145A and 145B. In other exemplary embodiments the gates 145A and 145B are before sensors S1-S4, as shown in FIG. 2. In other exemplary embodiments, gates 145A and 145B and sensors S1 and S2 are in staggered position.

The flow switch (gates) 145A and 145B is a valve or other device which may operate in at least an on/off state. The flow switch (gate) 145 may be a pinch valve such as, but not limited to, a NPV Series Miniature Pinch Valve commercially available from Clippard. The flow switch (gate) 145 is configured to respond at frequencies between 1 hz and 1000 hz, but primarily between 100 hz and 1000 hz. The response time of the flow switch (gate) 145 may be between 200 ms and 700 ms. Thus, for very low flow rates, the gate 145 may require its frequency to be lowered from 100 hz to 10 hz or lower (in some cases 1 hz), while running at pulse widths of between 200 ms to 700 ms.

The in-line flow rate sensor(s) S may be formed from any structure capable of sensing flow rate of the infusion fluid in the tubing of the administration set in real-time and sending the flow rate feedback to the controller unit 135. According to some exemplary embodiments of the invention, the sensor S measures viscosity to accurately determine direct flow rate. More specifically, in some exemplary embodiments, the in-line flow rate sensor is a thin nano wire of platinum surrounded by thin layer of parylene, which is an FDA approved coating to isolate the infusion fluid from any electric effects. This configuration of the sensor is sensitive to deflection, which is nearly directly proportional to flow rate once compensated for temperature and viscosity. In one exemplary embodiment, the infusion system uses a single sensor with gating method to determine viscosity, air inside the line, and flow rate. In one exemplary embodiment, the infusion system uses a two gated system and algorithm to determine viscosity, air inside line, flow rate and error correcting/confirming flow rate. Kinematic viscosity may be determined using a pulse or gating method and measuring the settling time. An estimate of dynamic viscosity may be made with the use of two sensors S1 and S2 in series as shown in FIG. 1 using a known inlet tubing resistance by taking an initial reading of flow rate and then making further calculations of density and viscosity, as required. An advantage of the two-sensor systems outlined above is the ability to have two independent flow rate detectors for comparison and confirmation of flow rate, thus increasing patient safety. These sensors can be fabricated using silicon wafer solid-state techniques, which are reliable and can be manufactured in sufficient volumes to be cost-effective.

In another exemplary embodiment of an in-line flow rate sensor S, a spinning (water) wheel arrangement built into the dedicated extension set may be configured to assess flow rate. For example, the wheel may have magnetic sensors and Hall effect devices to sense the rotation of the wheel to calibrate flow rate into units of ml/hr.

In yet another exemplary embodiment, the in-line flow rate sensor S may be a platinum wire strain gauge that provides a resistance. The fluid flow rate produces a strain force proportional to the flow rate. The resistance of the strain gauge increases as the strain increases and decreases as the strain decreases. As flow rate in the administration set increases, the resistance of the platinum wire increases, and as flow rate in the administration set decreases, the resistance of the platinum wire strain gauge decreases. The measured resistance of the platinum wire strain gauge is fed back to the controller 135, and the controller 135 can change the fluid flow rate in responses to changes in the measured flow rate in the administration set.

In some exemplary embodiments of the invention, the infusion systems may incorporate a dual gating technique that interrupts the fluid flow with discrete fluid pulse modulations created by the controller 135 pulsing gates G1 and G2 open and shut, as described below. In one exemplary embodiment shown in FIG. 1, gate G1 145 is positioned between the infusion fluid reservoir 125 and sensors S1, S2, and gate G2 is positioned after sensors S1, S2 and before manifold/connector 120. Pulse modulations include a pulse repetition frequency of the infusion fluid, a duration of a pulse of the infusion fluid, a pulse width of the infusion fluid, and the period of a pulse of the infusion fluid. In another exemplary embodiment of the invention, gating a range of flow rates from very slow to very fast is performed by the pulse modulation. The two gates are spaced at a pre-determined distance apart to create very slow flow rates by opening the upstream gate with the downstream gate closed. Then, by opening the downstream gate and closing the upstream gate, a small amount of drug is released (and delivered) that was stored in the tubing between the two gates. This opening and closing of the gates results in the pulse modulation of the fluid flow. The gates are also used to shut off the in-line flow on the tubing. In some exemplary embodiments, the tubing includes a soft tubing area for these gates to act upon. In some exemplary embodiments, the gates use a positive toggle action switch which requires very low energy (electrical consumption) but decisively shuts off the fluid flow. The gates are governed by the controller computing unit 135, which incorporates feedback from one or more in-line flow rate sensors that detect the flow rate and send the appropriate gating commands to the gates to ensure the flow rate output is the flow rate desired. The feedback includes an initial viscosity measurement, which is used to determine direct flow rate, which may be then compared to set the flow rate (input in the user interface). The flow switches (gates) 145A and 145B may include any number of gating mechanisms including a valve or other electromechanical mechanisms that are controllable and may accurately generate the pulse modulations. The gates may be incorporated into the administration set and/or infusion pump to provide the pre-determined distance. For example, gate G1 may be positioned near the exit of the infusion pump to the administration set and the other gate G2 within the administration set, specifically in the extension set, near the manifold.

The needle sets 113 may be subcutaneous, pre-packaged needle sets that include a disc or butterfly 150, which partially encloses a length of the needles 140 along the administering ends, such as disclosed in more detail in the assignee U.S. patent application Ser. No. ______ entitled “Systems and Methods for Precision Matched Immunoglobulin Infusion” (Attorney docket no. 213.0001-US00) filed simultaneously herewith, the disclosure of which is incorporated by reference herein in its entirety. The butterfly 150 is connected in series and in the same direction as the length of the series tubing. The butterfly 150 houses the needle 140 such that the needle protrudes both orthogonally to the long axis of the butterfly and to the series needle tubing. In one aspect, the needle may be bent to achieve this orthogonality. Furthermore, the butterfly housings have symmetrically positioned butterfly wings. The butterfly wings are used as a needle insertion/removal handling feature and, conform to the patient's skin without causing irritation or discomfort. The butterfly wings also protect the needle after use to eliminate potential harm, (e.g., needle-stick injuries). To protect the needle after use, the butterfly wings use a closing mechanism around the length of the needle to enclose the needle tip. This closing mechanism includes a double latch, in which both butterfly wings have a latch configuration to mate with the opposing wing. When the butterfly wings are closing, users will observe a tactile and/or audible click indicating to users that the butterfly wings are closed, and the needle tip is protected (after use of the needle set). Furthermore, the surface topography of the butterfly wings and its closing mechanism to increase surface area and thus reduce discomfort and pain when placed on the skin. In addition, the surface topography of the butterfly wings, the closing mechanism acts as a guiding feature to guide the butterfly wings together when closing. This prevents misalignment and makes it easier to cover and protect the needle.

Furthermore, the butterfly 150 wings may include grooves designated to guide and maintain the needles' orthogonal (90°) orientation such that the needle is straight and undamaged when received by the user. This ensures that the needles do not fail to penetrate to the correct skin tissue depth as a result of an angled needle, and the associated discomfort and pain from improper penetration is eliminated.

Furthermore, the needle 140 can include a ball-and-pivot or floating ball feature such that when inside of the butterfly housing the needle can rotate (e.g., five degrees) in any direction at the pivot point within the butterfly housing. In this fashion, slight motion of the butterfly does not transmit to the needle and does not cause the needle to move within a patient's tissue. As a result, the needles 140 may eliminate motion forces transmitted through the needle during an infusion, which can otherwise damage tissue and cause pain and inflammation. The pivoting needle feature eliminates these issues by rotating the needle at the pivot and within the butterfly housing in response to forces placed on the butterfly such that motion resulting from forces acting upon the butterfly is translated to the skin around the butterfly-to-skin contact area instead of the forces translating to the needle, which may damage tissue and cause pain and inflammation.

### Fluid Flow Controller

The fluid flow controller (a.k.a. micro controller computing unit) 135 is a programmable logic controller (PLC) or other known programmable processing device and may be incorporated into either of the infusion pump 105 (as illustrated in FIG. 1) or administration set 101. In some exemplary embodiments, the fluid flow controller 135 may be a separate part of the infusion system 100. In one exemplary embodiment, the fluid flow controller 135 receives parameters input by a clinician/technician, including a pre-set maximum pressure, a pre-set resumption pressure, and a pre-set flow rate. The controller modulates the flow rate, including reducing the flow rate to zero, to maintain the actual infusion fluid pressure within an acceptable range. For example, the user can control the flow rate in this on-off mode using a pulse modulation scheme, or the flow rate can be altered as a function of the deflection on the sensor. That is, the flow rate is regulated/modulated based on lower and higher infusion fluid pressures as they occur during the infusion time. As long as the infusion fluid flow rate results in a pressure below the pre-set maximum pressure, the flow rate will continue. For example, the flow rate can be pre-set to a maximum allowable flow rate and can be based on a maximum pressure. In some embodiments of the invention, the pre-set maximum pressure is between about 50 mmHg and about 800 mmHg, or between about 300 mmHg and about 750 mmHg. In some example embodiments of the invention, the pressure of the infusion fluid is maintained at 13.5 psi (698 mmHg).

The flow rate resistance measure can be displayed visually or audibly presented, such as a (site) pressure read-out or a continuous audible signal, respectively, when the pressure is within or outside the pre-set range. The patient or clinician can monitor the measurements to determine and/or confirm that the infusion fluid is being delivered correctly. The controller can record and track performance parameters for future review and correlation. Upper pressure limits can be pre-defined to ensure that excessive pressures are not reached.

In one exemplary embodiment, as illustrated in FIG. 1, the fluid flow controller 135 display information on a LCD display or other user interface or digital display 165 for presentation of at least one of fluid flow, determined data associated with fluid flow, and gate state information. The fluid flow rate controller 135 receives flow rate feedback from sensors S1 and S2 and provide control to gates 145A and 145B and actuator 115 of infusion pump 105. Similarly, the sensors S1-S4 of FIG. 2 also send signals indicating force due to fluid flow to the controller 135.

In the exemplary embodiment of FIG. 1, one or more needles 140 are configured in a parallel configuration. The administration set 101 can include a connection device 130, such as a Luer lock, for example. The administration set 101 includes an extension set 160 that extends from the Luer lock 130 to a manifold 120 which divides into individual needle tubes 110 and needles 140 to form the needle set (i.e., the tubes and needles extending from the manifold 120) 110. For convenience and brevity, a three-needle needle set 113 are shown in FIG. 1, but additional needle sets 113 with needles 140 can be used. For example, between one and eight needles 140 for subcutaneous use. Similarly, additional disks or butterfly assemblies 150 can also be used with each needle 140 placed on its own disk 150 FIG. 1. In another exemplary embodiment of the invention, the administration set 101 only includes an extension set 160 for intravenous use that may be connected to an indwelling catheter within the patient.

As further shown in exemplary configuration of FIG. 1, an in-line flow rate sensor S1 and flow rate sensor S2 are positioned in administration set tubing 160 between the Luer lock connection device 130 and manifold 120. The flow rate sensors S1 and S2 are a predetermined distance from each other within the tubing. There is also a predetermined distance between the infusion reservoir dispensing end 125 and flow rate sensor S1. The predetermined distances between components is used in determining fluid viscosity and flow rate. The distance between sensors S1 and S2 may be predefined to be between about 10 cm and about 40 cm based on the average length of extension sets 260 for common treatment uses. The fluid passes an air elimination filter 154 that removes air from the system before reaching the flow rate sensors. In operation, infusion pump 105 delivers pulses of the infusion fluid (e.g., 0.5-1.0 μL) to a patient over a short pulse duration at regular intervals as is known in the art. The pulse duration can be 50 milliseconds, 1 second, 5 seconds, etc., and the intervals can be over a period of 1 minute, 10 minutes, 1 hour, and other regular intervals. The infusion system 100 includes an infusion pump 105, infusion pump controller 135, actuator 115, infusion fluid reservoir 125, flow switches (gates) 145A and 145B, and the administration set 100 as described above. In operation, infusion pump controller 135 delivers a pulse of infusion fluid from the infusion reservoir 125 to a patient via a fluid path (FP) from the infusion pump 105 through the administration set 100. Via a user input 165 operational parameters are entered into controller 135 as described above. Controller 135 initiates the flow of the infusion fluid by engaging actuator 115 that forces infusion fluid from infusion reservoir 125 through flow switch (gate) 145A into administration set 101 along flow path FP.

As noted above, the in-line sensors may take the form of a nano wire of platinum coated with a thin layer of parylene, a spinning wheel sensor, and a platinum wire strain sensor as described above or other in line sensors capable of receiving and measuring characteristics of the infusion flow (pulse) to determine the flow rate and viscosity of the infusion fluid in the administration set, which can be used to calculate pressure at the patient's anatomic site. In-line sensors S1 and S2 send the flow rate feedback to the controller 135, which calculates the flow rate and length of time of the infusion fluid in the administration set 101. Controller 135 controls flow switch (gate) 145A to after and restrict the flow of the infusion fluid into the administration set 101 when the determined flow rate exceeds a predetermined setting or is outside a predetermined range of acceptable flow rates. Controller 135 can control flow switches (gates) 145A and 145B to modulate the pulse of the infusion fluid flowing from infusion fluid reservoir 125, including changing pulse width, the pulse repetition frequency (PRF), the duration of the infusion fluid pulse, and/or the period of the infusion fluid pulse by opening and dosing the upstream and downstream control flow switches 145A and 145B respectively to generate the fluid pulse. For example, opening the upstream switch 145A and dosing the downstream switch 145B to slow/stop the fluid flow to the patient. Then, dosing the upstream switch 145A and opening the downstream switch 145B to release a pre-determined amount of the fluid to the patient. As discussed, the duration and period of such pulses regulates and provides a direct indication of the (site) pressure of the infusion fluid for maintaining safe levels.

In the exemplary embodiment illustrated in FIG. 1, a fluid reservoir 125, such as a syringe is placed in the infusion pump 103. A user may then enter a desired flow rate into the user interface 165. The controller 135 commands both gates 145A and 145B to full open, and fluid from the reservoir 125 begins to move through the infusion system 100. When the fluid is first sensed at sensor S1 a signal is sent that the fluid has arrived at the sensor S1. The controller 135 then begins a timer noting the time of receipt of a second signal when the fluid reaches sensor S2. The time it takes to go from sensor Si to sensor S2 is known, and with uniform inlet tubing on every administration set 101, a determination of the fluid viscosity may be made. In some exemplary embodiments, the fluid viscosity may also be manually entered into the user interface 165 for the controller to make any flow rate determinations.

Once the fluid viscosity is known, sensor S1 may provide the flow rate of the fluid to the controller 135. The controller will begin to limit the flow rate from wide open and determine the right combination of pulse width and frequency to deliver the programmed/entered fluid flow rate. If the controller is reading a flow rate lower than the desired value, it will increase pulse width or frequency to increase the rate. For very slow fluid flow rates, it is likely that the pulse repetition rate itself will manage the flow rate to maintain higher accuracy at low fluid flow rates. Concurrently, sensor S2 is monitoring and sending the flow rate to the controller 135 for comparison. When the sensors S1 and S2 disagree, the system 100 will shut down presenting a flow rate error.

When mid-range flow rates are selected, use of either gate 145A or 145B may result in enough control for the desired flow rate to be met. However, for very low flow rates, the use of both gates 145A and 145B may be necessary to meet the desired flow rates. In other words, for low flow rates, as described above, gate 145A is turned on (opened), while gate 145B is turned off (closed). Then gate 145A is closed and gate 145B is opened, allowing the residual pressure between the gates to drive the fluid forward, very slowly. This process can be repeated as quickly or slowly as needed. This method has the advantage of dispensing lower volumes of drug at more frequent intervals, which may minimize potential for blood clotting within the patient. Alternatively, very low flow rates can also be realized using the shortest pulse width required for minimum gate(s) response and by extending the time between pulses, as necessary. This method, however, may result in long time periods between dispensing volumes of drug, which may increase the risk of clots within the patient or administration set. One advantage of this two-gate 145 system is if the controller 135 starts seeing a flow rate going faster than was desired, and is sending signals to gate 145A which is not responding appropriately, that would indicate a failure of gate 145A, and the second gate 145B can then be used to either control the fluid correctly or shut down the system 100 due to gate 145A failure, again resulting in greater safety for the patient.

To determine pressures at an infusion site of a patient, the system 100 determines, based on a known inlet pressure of the infusion pump 105, e.g., 13.5 psi. The controller 135 generates a pulse modulation that meters the flow out at the desired flow rate, e.g., 60 ml/hr. Once set, the controller 145 does not change the pulse modulation. If, a sensor 145A or 145B records a decrease in flow rate to, e.g., 50 ml/hr. Because the inlet pressure is known, and the system 100 can assume that the starting pressure at the infusion site was zero, then the decrease of 10 ml/hr is solely caused by the infusion site increasing in pressure. The controller 135 can then calculate the internal pressure required to return the infusion site pressure to its previous state by reducing the flow rate using the Hagen Poiseuille equation.

Additionally, infusion site pressure may be determined by changes in the pulse modulation, as made by the controller 135. The controller 135, may need to increase the pulse modulation to maintain the desired flow rate, based on the amount of change required, the controller 135 may also determine what the likely increase in internal resistance should be.

In some exemplary embodiments, the infusion can continue until the fluid is exhausted at which time both sensors S1 and S2 will record a lack of flow rate. In other exemplary embodiments, since the controller 135 knows the flow rate and the infusion time, it can also calculate volume of infusion fluid used. If the initial volume is entered into the system 100 (or detected, for example, in a syringe-based system), then the controller 135 may provide an alarm indicating either a set volume of infusion fluid has been met, or an exhaustion or near-exhaustion of the infusion fluid is completed.

In some exemplary embodiments, air pockets in the infusion system 100 may be determined by the sensors S1 and S2. When air passes through the sensors S1 and S2, a spike in flow rate is sensed.

FIG. 2 illustrates another exemplary embodiment of infusion system 200 constructed according to an exemplary embodiment of the invention for delivering immunoglobulins to patients as part of their infusion therapy. The infusion system 200 includes an infusion pump (e.g.., driver) 105, which is connected to an administration set 201. Administration set 201 in this exemplary embodiment includes a needle set 213 including a manifold/connector 120, needle tubes 210, needles 140, and flexible butterfly assemblies 150. In-line sensors in each of the needle tubes 210 that may take the form of any one or more of the sensor structures described above. The flow rate sensors S1, S2, S3, and S4 are spaced a predetermined distance from each other within the tubing. There is a defined distance between flow rate sensor S1 in the extension set 160 and flow rate sensors S2, S3, and S4. In each of the needle tubes 210. There is also a pre-defined distance between the infusion reservoir dispensing end 125 and flow rate sensor S1. The predetermined distances between components is used to determine fluid viscosity and flow rate.

The fluid passes an air elimination filter 154 to remove the air from the system before the air reaches the flow rate sensors S1-S4. In this configuration, the characteristics of the infusion flow (pulse) is measured in each of the needle tubes 210 using in-line sensors S2-S4 to determine the flow rate of the infusion fluid in each of the needle tubes 210, and flow rate feedback is provided to controller 135 as above which may benefit is a device diagnostic tool and provide improved flow accuracies. The improved flow accuracies are realized by determining flow in each of the needle tubes 210 rather than a global flow rate of the extension set 160 of the administration set 201 up to the manifold 120. Determining flow rates in the needle tubes 210 provides additional flow rate data and improved accuracy. If any of the in-line sensors S1-S4 provides a measurement indicative of a flow rate outside the range of the desired flow rate, controller 135 controls flow switch (gate) 145A to after and restrict the flow of the infusion fluid into the administration set 201. As before, controller 135 can control flow switches (gates) 145A and 145B to modulate the pulse of the infusion fluid flowing from infusion fluid reservoir 125 by opening and dosing the upstream and downstream control flow switches 145A and 145B respectively to generate the fluid pulse. The opening and dosing of the switches 145A and 145B control the pulse repetition frequency (PRF) or pulse width of the infusion fluid pulse, the duration of the infusion fluid pulse, and/or the period of the infusion fluid pulse. In this manner, the flow rate of the infusion fluid can be measured and maintained at safe levels.

In-line flow rate sensors constructed according to the principles and exemplary embodiments of the invention may be used with various other drivers, including the drivers and systems described below.

Gravity Controlled Drivers: According to one exemplary implementation of the invention, a gravity control intravenous system without full gating control uses the flow rate sensor(s) constructed according to the principles and some exemplary embodiments of the invention to estimate and display the flow rate. Flow rates may be set by a simple mechanical flow rate device such as, but not limited to, a Dial-A-Flow device commercially available from Wolf Medical Supply, or other gravity flow regulators commercially available from Abbott Laboratories and Baxter International, and the flow rate may be displayed accurately on a liquid crystal (LCD) screen or other display device. If the flow rate exceeds minimums or maximums, an alarm system may be activated. This exemplary embodiment of the invention is a low-cost option that assumes that most intravenous (IV) fluids used have densities that are close enough to water to measure the flow rate solely by the deflection mechanism of the platinum nanowire sensor. This exemplary implementation produces a low-cost and simple system for IV delivery. Using the double gating and double sensing features constructed according to the principles and exemplary embodiments of the invention, a fully controllable gravity system can precisely control the flow rate, and can operate from gravity pressures of 1.2 psi up to almost any pressure pump level as needed.

Elastomeric Pumps: Elastomeric pumps operate in a range from 3 psi to about 7 psi and have varying pressure profiles. As outlined above, these pumps are inherently inaccurate. Using features constructed according to the principles and some exemplary embodiments of the invention, the elastomer pump may have a dedicated administration set that clamps off the fluid flow and automatically releases when the administration set is used with the flow controller. The flow controller may create accurate flow rates for a wide range of medications using pulse width and rate control using a microprocessor of the flow controller. The microprocessor may incorporate feedback of the flow rate sensor(s) and input(s) from the keypad to pre-set the flow desired flow rate. With separate control inputs, such systems can also deliver programmed bolus rates for patient-controlled analgesia (PCA). A wide range of flow rates is achievable with these systems but there will be some limits to maximum flow rates due to the lower pressures associated with these devices. Operationally, the elastomer is filled in the pharmacy and sent to the patient with attached dedicated flow control tubing that is shipped both in a “no-flow” condition as well as having a non-vented luer cap for additional protection against drug outflow. When the administration set is use with the flow controller, the controller assumes flow control of the gates and full control of the flow rate can be reached with inputs from the keypad on the controller.

Constant Pressure Systems (CPS): Systems that operate at a constant pressure have some advantages over other systems, because pressure increases at the patient site decrease the flow rate unless an appropriate adjustment is made. When a CPS infusion driver is connected to a controller constructed according to the principles and some exemplary embodiments of the invention, a pulse modulation of the fluid flow may be determined to achieve a desired flow rate, if a blockage or tissue saturation occurs, and if the pulse modulation coding remains unchanged, the flow rate automatically decreases. This is immediately detected by the flow sensors, and an indication of a blockage or tissue saturation is generated on a user display and an alarm indicated.

For example, CPS systems constructed according to the principles and some exemplary embodiments of the invention may include modifying a pulse waveform. The pulse waveform is modified to maintain the desired fluid flow rate. This also increases the site pressure as the flow switches (e.g., gates) are open longer and more frequently, approaching wide open. This modality may be used for drug studies, where a drug manufacturer wants to maintain a known flow rate. In some exemplary embodiments, if desired, an indication of increased fluid flow, and thus drug delivery, is noted in a user display and included as an alarm. This provides several benefits including: constant flow (depending on a maximum pressure) for increasing tissue site resistance but within safe maximum pressures or dynamic equilibrium as long as a safe maximum pressure is selected by the controller. The advantages of such systems include safety, flexibility, versatility, a wide range of flow rates, detection of air, a known flow rate delivery at all times, and competitive costs for all of these benefits. The sensor arrangements and gating methods for this exemplary implementation may be the same or similar to the configurations discussed above.

For subcutaneous delivery of immunoglobulins there are additional benefits of the CPS systems constructed according to the principles of the invention. These are often of great importance to clinical trials and may find their way into clinical practice. For example, by placing a sensor into each leg of a multi-needle infusion set, such as disclosed, e.g., in FIG. 2, the infusion system determines the flow rate delivered to each site separately. This may be accomplished by multiplexing and sampling signals from the sensors and can be thought of as having a separate fluid flow controller for each site (leg). Using this configuration, the infusion sites that have the best absorption are identified. Further, the system determines the actual site pressure of each location by knowing the steady pressure delivered by the CPS and the initial flow rate. As the site(s) pressures increase, because of dynamic equilibrium, the flow rate decreases. Further, these infusion systems calculate the site pressure necessary to reduce the flow rate by the measured amount. If the infusion source varies in pressure, these parameters may be determined by adding a (solid state) pressure sensor into the sensor array to monitor instantaneous pressure at the source to determine the internal pressures of the delivery inside the patient's anatomic space. This pressure determination is made without requiring any pressure transducers, wires, or other methods being invasively implanted into the patient.

Variable Pressure Systems (VPS) and Coiled Spring Pumps: These types of infusion drivers have a varying pressure as a function of volume. Normally, the flow rate starts high and decreases as the fluid is consumed and the pressures drop. If used for the delivery of subcutaneous immunoglobulins (e.g., SCIg), there is an increase in the time of delivery based on the volume of drug infused. This can be misidentified as tissue saturation, which also increases the time of delivery. In existing systems there is no way to determine the actual cause of the increased time. VPS systems constructed according to the principles and some exemplary embodiments of the invention may measure the first half of infusion with a flow rate of 60 ml/hr. The infusion system may check to ensure that within 30 minutes, 30 ml volume of drug was released. These infusion systems may then measure the second half of the infusion with the same expected 60 ml/hr flow rate. However, the time length of the infusion will typically be longer than the expected 30 minutes. This change in infusion rate is characteristic of conventional VPS's varying pressure as a function of volume. However, using a VPS system constructed according to the principles of the invention can eliminate many of these shortcomings. Since these sensors can determine and set the actual flow rate to any flow rate desired, the change in pressure with volume does not change the flow rate. If the curve of pressure reduction with volume can be identified for the infusion driver, a VPS system constructed according to the principles of the invention can make the determination if the pulse compensation is due to increased back pressure at the patient's anatomic site. Otherwise the VPS system can be controlled for constant flow rate, but the varied pulse modulation required will not necessarily be indicative of complications at the site. Alternatively, an inline pressure sensor inside the sensor array could adjust for the varying source pressure as described above.

Infusion systems and methods constructed according to the principles and exemplary embodiments of the invention may accurately and reproducibly deliver an infusion fluid to a patient at a desired anatomical space by allowing for direct real-time monitoring and control of the infusion system pressure. Exemplary embodiments of the invention include a flow measurement system built into the administration set tubing to accurately and instantaneously measure flow rate and viscosity of the infusion fluid in the tube(s) of the administration set and to provide immediate feedback to the infusion pump to control flow rate parameters. Patients and clinicians can determine the infusion system flow rate and deliver a volume of an infusion liquid at a speed (e.g., flow-rate) that does not cause discomfort. The flow rate and pressure can be set to minimize the amount of pain or discomfort caused by resistance of fluid-filled tissue space and associated pressures during infusion therapies.

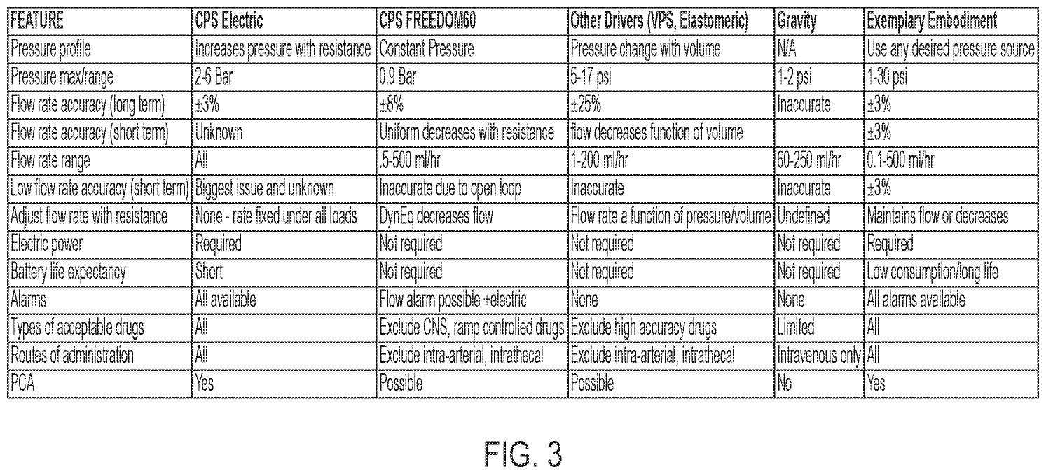

FIG. 3 summarizes the features and advantages of an exemplary embodiment of an infusion system constructed according to the principles of the invention in comparison to certain conventional systems. In conventional systems, an infusion fluid flow rate is set, for example to a flow rate of 20 ml/hr. As the infusion therapy begins and the infusion fluid is delivered to the patient, flow resistance increases, while the flow rate monitor continues to read 20 ml/hr. With the increased flow resistance, the infusion pump must increase pressure to continue to deliver the infusion fluid at 20 ml/hr. As the infusion therapy session progresses, the flow resistance continues to increase, and the infusion pump continues to increase pressure to meet the target 20 ml/hr flow rate. At some point in the infusion therapy session, the pressure becomes so high that an occlusion alarm goes off indicating an unsafe pressure level in the system.

In conventional electrical infusion pump systems, the pressure of the infusion fluid in the flow path from the infusion reservoir to the patient is determined by measuring the force applied to a piston using a transducer or other infusion flow actuation of the infusion pump or by monitoring the electrical current going to the infusion pump driving motor. The pump force includes static and dynamic friction forces associated with the pump/piston actuator in addition to the pressure in the fluid path. That is, for syringes for example, the actuator force applied includes a combination of static friction, dynamic friction, and mechanical forces in addition to infusion fluid pressure. The infusion fluid pressure may make up only a small part of the overall force applied to the actuator. As such, the measured force of the actuator may not be an accurate representation of the infusion fluid pressure in the administration set. To adjust the flow rate in response to a blockage within the patient's anatomic space a force transducer responding to a change in load or a motor responding to a change in current may be used. In the case of blockages, the pressure (force applied to the actuator) must increase to achieve the preset flow rate. As a result of the administration set tubing length, size, and mechanism the pressure buildup is slow. In the event of an occlusion at that patient's anatomic site, the pressure at the pump may be too high (in order to achieve the set flow rate), as such a high pressure is delivered to the patient's anatomic site which may cause patient harm. That is, the driver's measured pressure is not indicative of the pressure at the patient's anatomic space. Relying on this method for assessing pressure at the patient's anatomic site can cause patient harm. At lower infusion fluid pressures in the administration sets, the friction forces of the actuator on the infusion fluid reservoir may dominate, and as a result, multiple actuation pressure measurements may be needed to determine that the infusion fluid pressure has increased to a point indicative of an occlusion.

In these systems, a blockage or other event may not be detected or may take too long to be detected to provide a safe delivery system for the patient. For example, if an insulin infusion results in an occlusion that goes undetected, the patient may not receive the proper dose of insulin to prevent a hyperglycemic event. In chemotherapeutic treatments, predictable and accurate infusion fluid delivery is essential. In-line flow rate sensors constructed according to the principles and exemplary embodiments of the invention detect flow rate of the infusion fluid in the administration set in real-time and can provide appropriate remediation, including altering the flow rate characteristics of the infusion fluid and/or sounding an alarm.

Exemplary embodiments of the invention include a flow rate sensor that receives real-time feedback and sends the feedback to a controller to meter the infusion fluid flow to compensate for and address increases in flow rate resistance in the system. The controller may meter the infusion fluid flow by pulsing or otherwise interrupting the constant fluid flow to deliver a pulsed frequency modulation of the fluid flow via flow switches (gates). The pulsed modulation of the fluid flow can be used to change the flow rate while continuing to deliver infusion fluid to the anatomical site. The gates may be used, via pulse modulation, to create a flow rate resistance that can be lowered until an acceptable pressure level is reached, and the patient can accept a delivered volume of an infusion liquid at a speed that does not cause discomfort. For example, if fluid flow is decreasing (thus resulting in higher pressure), the gates may open up more (i.e., decreasing flow rate resistance) until the system determines an acceptable pressure at the infusion sites is achieved. With the infusion systems constructed according to the principles and exemplary embodiments of the invention, a patient or clinician can set pressure and flow rate variables to minimize the amount of pain or discomfort caused by resistance of fluid-filled tissue space and associated pressures. In other words, the clinician can set internal site pressure limits to accurately and automatically reduce the flow rate to limit any internal damage.

Similarly, for subcutaneous administrations, a patient or clinician can use administration sets with multiple needles (e.g., from 2-8 needles) in parallel to infuse the patient at multiple sites to decrease the overall infusion time. In embodiments of the invention, no change in flow rate and no change in pressure needs to occur.

Infusion systems constructed according to the principles and exemplary embodiments of the invention can use a predetermined maximum pressure to automatically prevent continuous drug (fluid) flow into surrounding tissues when the system internal pressure exceeds the maximum or can use a range of flow rates to meter fluid flow during an infusion. The flow rate limits and acceptable pressure ranges limit the delivery of an infusion fluid into unintended tissues by eliminating the need for a continuous flow of fluid into saturated sites or tissues. When the site pressure is lowered during the perfusion, the flow resumes and continues within the flow rate limits.

Although certain exemplary embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

The invention claimed is:

1. An administration set for delivering an infusion fluid into a patient's anatomic space, the administration set comprising:
an infusion fluid path having a first portion to connect with a source of the infusion fluid and a second portion to deliver the infusion fluid into the patient's anatomic space; and
a flow rate sensor disposed in the infusion fluid path between the first and second portion to determine a flow rate measurement of the infusion fluid and to generate a signal indicative of the flow rate measurement to control the flow rate of the infusion fluid through the fluid path.

2. The administration set of claim 1, wherein the sensor comprises two flow rate sensors spaced apart a predefined distance within the infusion fluid path.

3. The administration set of claim 2, further comprising:
an extension set; and
a needle tube, and
wherein one of the flow rate sensors is disposed in the extension set and another flow rate sensor is disposed in the needle tube, with the sensors being spaced apart a predefined distance.

4. The administration set of claim 1, further comprising a second flow rate sensor, and
wherein the flow rate sensors are disposed in the infusion path and are spaced apart a predefined distance within the infusion fluid path.

5. The administration set of claim 1, wherein the sensor is selected from one of the group of a nano wire of platinum coated with a thin layer of parylene, a spinning wheel sensor, and a platinum wire strain sensor, and the flow rate measurement comprises a resistance measurement.

6. The administration set of claim 1, further comprising:
a controller to receive the signal indicative of the flow rate measurement from the flow sensor, and parameters input by a user, wherein the parameters include at least one of the group of a pre-set maximum pressure, a pre-set resumption pressure, and a pre-set flow rate; and
a fluid flow gate to control the flow rate of the infusion fluid through the fluid path.

7. The administration set of claim 6, wherein the controller is a programmable logic controller configured to operate the fluid flow gate to control the flow rate of the infusion fluid through the fluid path in a pulsatile manner.

8. The administration set of claim 6, wherein the fluid flow gate comprises at least two gates configured to turn on and off, and the flow rate sensor disposed in the infusion path is configured to generate a signal indicative of the flow rate measurement to enable the controller to determine at least one of the group of a viscosity, a presence of air inside the fluid path, and a flow rate and error correction.

9. The administration set of claim 6, wherein the signal received from the sensor is used to determine internal pressures inside the patient's anatomic space to limit adverse site reactions.

10. The administration set of claim 6, wherein the controller is further configured to generate an alarm signal when the flow rate exceeds a maximum flow rate, or when the flow rate fails to meet a minimum flow rate, or when the controller acts upon the fluid flow gate to stop or slow flow of the infusion fluid for patient safety.

11. An infusion system for delivering an infusion fluid into a patient's anatomic space, the system comprising:
a pump to drive infusion fluid from a source of the infusion fluid into the patient's anatomic space; and
an administration set including:
an infusion fluid path having a first portion fluidically connected to the pump and a second portion to deliver the infusion fluid into the patient's anatomic space; and
a flow rate sensor disposed in the infusion fluid path to determine a flow rate measurement of the infusion fluid and to generate a signal indicative of the flow rate measurement to control the flow rate of the infusion fluid through the fluid path.

12. The infusion system of claim 11, further comprising:
a fluid flow gate to control the flow rate of the infusion fluid through the infusion fluid path; and
a controller to control at least one of the group of the pump and the fluid flow gate based upon the signal from the flow rate sensor, and wherein the controller is configured to modulate the flow rate of the infusion fluid including to reduce an instantaneous flow rate to zero or to maintain an actual pressure of the infusion fluid within a predetermined acceptable range.

13. The infusion system of claim 11, wherein the controller is configured to modulate a flow rate of the infusion fluid based on the signal using a pulse width modulation to create an off/on cycle to control the flow rate of the infusion fluid, and wherein the pulse width includes a time in which the infusion fluid is flowing at a non-zero flow rate to maintain an actual pressure of the infusion fluid within a predetermined acceptable range.

14. The infusion system of claim 11, wherein the in-line flow rate sensor comprises at least one of the group of a nano wire of platinum coated with a thin layer of parylene, a spinning wheel sensor, and a platinum wire strain gauge, and the flow rate measurement comprises a resistance measurement.

15. The infusion system of claim 11, further comprising:
an extension set; and
a needle tube, and
wherein the flow rate sensor comprises two flow rate sensors, with one of the flow rate sensors disposed in the extension set and another flow rate sensor disposed in the needle tube, with the sensors being spaced apart a predefined distance.

16. The infusion system of claim 11, further comprising:
a second flow rate sensor, and
wherein the flow rate sensors are disposed in the infusion path and are spaced apart a predefined distance.

17. The infusion system of claim 11, wherein the pump comprises an infusion driver selected from one of the group of a constant pressure system, elastomeric pump, gravity system, coil spring pump, variable pressure pump, and electrically powered pump.

18. A method for administering an infusion fluid into a patient's anatomic space via an infusion fluid path, the method comprising:
receiving an in-line flow rate measurement from an in-line flow rate sensor in the infusion fluid path;
modulating a flow rate of the infusion fluid into the patient's anatomic space to maintain the in-line flow rate measurement within a predefined acceptable range.

19. The method of claim 18, further comprising:
determining at least one of the group of a viscosity, a presence of air inside the fluid path, and a flow rate; and
error correcting to maintain the flow rate.

20. The method of claim 18, further comprising:
determining, based on a signal from the sensor, internal pressures of the patient's anatomic space; and
adjusting the flow rate of the infusion fluid to limit adverse site reactions.

21. The method of claim 18, wherein the step of receiving the in-line flow rate measurement comprises receiving a resistance measurement from at least one of the group of a nano wire of platinum coated with a thin layer of parylene, a spinning wheel sensor, and a platinum wire strain gauge.

22. The method of claim 18, wherein modulating the flow rate of the infusion fluid into the patient's anatomic space comprises:
changing at least one of the group of a pulse repetition frequency of the infusion fluid, a duration of a pulse of the infusion fluid, a pulse width of the infusion fluid, and a period of a pulse of the infusion fluid.

23. The method of claim 18, wherein the infusion fluid comprises a chemotherapeutic, antibiotic, or an immunoglobulin.
